(19) United States
(12) Patent Application Publication
(10) Pub. No.: US 2003/0195272 A1
(43) Pub. Date: Oct. 16, 2003

(54) Scented hot melt adhesives

(76) Inventors: Michael Harwell, Hillsborough, NJ (US); Diane Montgomery, New South Wales (AU); Leisa Ryan, Ashfield (AU)

Correspondence Address: Cynthia L. Foulke, Intellectual Property, NATIONAL STARCH AND CHEMICAL COMPANY, P.O. Box 6500, Bridgewater, NJ, 08807-0500, US

(21) Appl. No.: 09/775,045

(22) Filed: Feb. 1, 2001

(51) Int. Cl. A61L 2/00

(52) U.S. Cl. 523/102

(57) ABSTRACT

Hot melt adhesives comprising an effective amount of a non-encapsulated fragrance is useful in the manufacture of absorbent articles, such as diapers/training pants and feminine hygiene products. The scented material has a closed cup flash point of greater than 100° F., most preferably a closed cup flash point of greater than 200° F.

### FIELD OF THE INVENTION

The present invention relates to scented adhesives. In particular, the invention relates to hot melt adhesives comprising non-encapsulated scents, to the use of a scented hot melt adhesive to prepare a disposable absorbent product and to disposable absorbent articles comprising scented hot melt adhesives.

### BACKGROUND OF THE INVENTION

Disposable absorbent products currently find widespread use in many applications. For example, diapers, training pants, incontinent garments, feminine hygiene pads, i.e., catamenial pads, sanitary napkins, panty liners, panty shields and the like, underarm shields and health care products such as surgical drapes or wound dressings. Such articles are designed to absorb body fluids, such as urine, menses, blood, perspiration and other excrements discharged by a body.

A typical disposable absorbent product generally comprises a composite structure including a fluid-permeable topsheet, a fluid absorbent core and a fluid-impermeable backsheet. These disposable absorbent products, when appropriate, usually include some type of fastening system for fitting the product onto the wearer.

Adhesives, often referred to as construction adhesives, are generally used to join the different parts of the disposable absorbent product together. Hot melt adhesives are preferably used for this purpose since such adhesives allow for cost and time efficient manufacturing since there is no evaporation step necessary as is the case for water-based or solvent-based adhesive systems.

While use of fragrances for odor control in sanitary disposable products, such as diapers and feminine hygiene products, has been addressed in the art, the incorporation of fragrances within the hot melt adhesive component used to construct such products has proved difficult. Effective perfuming of hot melt adhesives, i.e., wherein the fragrance remains stable during production, during storage prior to use, and is still available for delivery after the adhesive has been used, has been difficult to achieve and a need remains in the art for a fragrant hot melt adhesive that can be used to prepare disposable absorbent articles, in particular disposable absorbent garments such as diapers.

### SUMMARY OF THE INVENTION

It has now been discovered that certain fragrance compositions may be added to hot melt adhesives to provide fragrance to nonwoven fabrics.

This invention is directed to hot melt adhesives having contained therein molecules of at least one fragrance composition capable of evolving from said adhesive into the environment proximate said adhesive.

One aspect of the invention is directed to a hot melt adhesive comprising a non-encapsulated scented material. Most preferably the scented material has a closed cup flash point of greater than 200° F. In one embodiment of the invention, the hot melt adhesive comprises “Spicy Oriental Amber Powder” (Quest Fragrance Q-26257) fragrance.

Another aspect of the invention is directed to a disposable absorbent article of manufacture comprising a liquid-permeable topsheet, a liquid-impermeable backsheet, a fluid-absorbent core material positioned between the topsheet and the backsheet and a hot melt adhesive containing a nonencapsulated scented material. A particularly preferred aspect of the invention is directed to disposable absorbent garments such as, for example, diapers for use in infants and toddlers, adult incontinent pads and feminine hygiene products.

Still another aspect of the invention is directed to a process for bonding a tissue or a nonwoven substrate to similar or dissimilar substrates in the construction of a disposable absorbent product comprising applying to at least one substrate a molten hot melt adhesive composition and bonding the substrates together, wherein the hot melt adhesive comprises a non-encapsulated scented material.

### DETAILED DESCRIPTION OF THE INVENTION

The invention provides fragranced compositions and a process for the effective perfuming of hot melt adhesives. The fragranced compositions may be added to hot melt adhesives without the a requirement for encapsulation or other protective means, and are capable of remaining stable in the adhesives prior to and after use without significant loss of fragrance.

The term effective perfuming means the fragrance remains stable during production, during storage prior to use and is still available for delivery after the adhesive has been used. By delivery means the scent evolves from the adhesive into the environment proximate to the adhesive. By stable is meant that the fragrance remains noticeable following manufacture of the adhesive, following storage of the adhesive, following manufacture of an article formed using the adhesive, during storage of the manufactured article and during use of the manufactured article by the end user.

The terms perfume, fragrance and scent are used interchangeably throughout this application and mean a composition comprising one or more fragrance materials, and optionally a solvent, formulated to have certain useful fragrance characteristics. In most cases fragrance compositions are formulated to have a fragrance generally considered at least inoffensive and preferably pleasing to intended users of the composition or user of the article prepared with the adhesive. Fragrance compositions are used for imparting a desired odor to the skin and/or any product for which an agreeable odor is indispensable or desirable. Fragrance compositions are also used in products that would normally have an unattractive or offensive odor to mask the odor and produce an odor that is less unattractive or offensive. The (pleasing) fragrance characteristics may be the main function of the product in which the fragrance composition has been incorporated or may be ancillary to the main function of the product.

In one embodiment of the invention, a disposable absorbent product is provided, which disposable absorbent product comprises (1) a liquid-permeable topsheet, (2) a liquid-impermeable backsheet, which topsheet may be attached to the backsheet, (3) an absorbent structure positioned between the topsheet and the backsheet, and (4) an adhesive having desired fragrant properties.

The absorbent structure will typically comprise a nonwoven fabric. A nonwoven fabric is defined as an interlocking fiber network characterized by flexibility, porosity and integrity. The individual fibers used to compose the nonwoven fabric may be synthetic, naturally occurring, or a combination of the two. The individual fibers may be mechanically, chemically, or thermally bonded to each other. Nonwovens are used commercially for a variety of applications including insulation, packaging, household wipes, surgical drapes, medical dressings, and in disposable articles such as diapers, adult incontinent products and sanitary napkins. Tissue is a closely related material in which the individual fibers may or may not be chemically bonded to one another.

The adhesives according to the invention may be formulated for use as, e.g., positioning adhesives, core adhesives or elastic adhesives, and are particularly suitable for use in the manufacture or articles, including but not limited to disposable absorbent products, such as diapers, adult incontinent products, bed pads; sanitary napkins, and in other absorbent products, such as, bibs, wound dressings, and surgical capes or drapes, which are used to absorb a liquid, such as water and saline, and body liquids, such as urine, menses, and blood. The fragranced adhesive of the invention may be used to adhere the nonwoven or tissue to another substrate or component. The second substrate may be another nonwoven, tissue, or an unrelated material.

The adhesive may be used to attach the topsheet to the backsheet. Alternatively, the adhesive may be used to adhere either the topsheet or the backsheet to other components of the disposable absorbent product, such as tissue layers, leg flaps, fastening ears, tapes, or tabs, or other components typically used to construct a disposable absorbent product that are well known to one skilled in the art.

Those skilled in the art will recognize materials suitable for use as the topsheet and backsheet.

Exemplary of materials suitable for use as the topsheet are liquid-permeable materials, such as spunbonded polypropylene or polyethylene having a basis weight of from about 15 to about 25 grams per square meter.

Backsheets often used in disposable absorbent products are generally prepared from liquid-impermeable materials which function to contain liquids, such as water, urine, menses, or blood, within the absorbent core of the disposable absorbent product and to protect bedding and/or a wears' outer garments from soiling. Materials useful as a backsheet in a disposable absorbent product are generally impermeable to liquid but are permeable to vapor. Examples are liquid-impervious materials such as polyolefin films, e.g., polypropylene and polyethylene, as well as vapor-pervious materials, such as microporous polyolefin films, sometimes referred to as breathable films.

A particularly desirable backsheet material is a film comprising a polyolefin polymer such as a linear low density polyethylene and a filler. As used herein a “filler” is meant to include particulates and other forms of materials which can be added to the film polymer extrusion blend and which will not chemically interfere with or adversely affect the extruded film but which are able to be uniformly dispersed throughout the film. When the film is stretched during processing, the filler generally causes a network of holes to be formed in the film. Such holes are generally small enough to prevent the passage of a liquid, but are generally large enough to allow vapor to pass through the holes. Generally the fillers will be in particulate form and usually will have somewhat of a spherical shape with average particle sizes in the range of about 0.1 to about 7 microns. Both organic and inorganic fillers may be used in the practice of the invention provided that they do not interfere with the film formation process. Examples of fillers include calcium carbonate (CaCO₃), various kinds of clay, silica (SiO₂), alumina, barium sulfate, sodium carbonate, talc, magnesium sulfate, titanium dioxide, zeolites, aluminum sulfate, cellulose-type powders, diatomaceous earth, magnesium sulfate, magnesium carbonate, barium carbonate, kaolin, mica, carbon, calcium oxide, magnesium oxide, aluminum hydroxide, pulp powder, wood powder, cellulose derivatives, chitin and chitin derivatives.

Fragrance compositions which may be incorporated into hot melt adhesives in accordance with the invention are fragrances that have a closed cup flash point greater than about 100° F., preferably greater than about 150° F., more preferably greater than about 200° F. as determined by ASTM method D93-00 (Flash Point by Pensky-Martens Closed Cup Tester). Any fragrance or fragrances having a flashpoint greater that 100 may be used in the practice of the invention. Such fragrances are commercially available. An example of a fragrance which may be used “Spicy Oriental Amber Powder” commercially available from Quest (Quest Fragrance Q-26257), which has closed cup flash point of greater than 200° F. The fragranced composition(s) are added to the hot melt adhesive composition without the requirement for encapsulation or other protective means. The term non-encapsulated is used as conventional in the art and means that the fragrant material or composition is not enclosed in a protective cover or shell. The fragranced composition is formulated into the hot melt adhesive without the prior encapsulation thereof.

The fragrance compositions may be added to virtually any hot melt type adhesive. Particularly preferred for use in the practice of the invention are low application temperature hot melt adhesive formulations, i.e., formulations that can be applied at temperatures below about 300° F., more preferably at about 250° F. and down to about 200° F. Low application temperature hot melt adhesives are commercially available from National Starch and Chemical Company, Bridgewater, N.J.

Any base polymer suitable for use in formulating hot melt adhesives, as are well known to those skilled in the art may be used in the practice of the invention. Such polymers include amorphous polyolefins, ethylene-containing polymers and rubbery block copolymers, as well as blends thereof. Hot melt adhesive compositions based on ethylene/vinyl acetate copolymers, isotactic or atactic polypropylene, styrene-butadiene, styrene-isoprene, or styrene-ethylene-butylene A-B-A or A-B-A-B block copolymers or mixtures thereof may be used. In addition to the base polymer, the hot melt adhesive compositions of the invention may also contain tackifiers, oils and/or waxes as well as conventional additives including stabilizers, anti-oxidants, pigments and the like.

In more detail, the fragrance compositions may be added to adhesives based on rubbery block copolymers. These polymers include the block or multi-block copolymers having the general configuration: A-B-A or A-B-A-B-A-B- wherein the polymer blocks A are non-elastomeric polymer blocks which, as homopolymers have glass transition temperatures above 20° C., while the elastomeric polymer blocks B are butadiene or isoprene or butadiene isoprene which is partially or substantially hydrogenated. Further, they may be linear or branched. Typical branched structures contain an elastomeric portion with at least three branches which can radiate out from a central hub or can be otherwise coupled together.

The non-elastomeric blocks may comprise homopolymers or copolymers of vinyl monomers such as vinyl arenes, vinyl pyridines, vinyl halides and vinyl carboxylates, as well as acrylic monomers such as acrylonitrile, methacrylonitrile, esters of acrylic acids, etc. Monovinyl aromatic hydrocarbons include particularly those of the benzene series such as styrene, vinyl toluene, vinyl xylene, ethyl vinyl benzene as well as dicyclic monovinyl compounds such as vinyl naphthalene and the like. Other non-elastomeric polymer blocks may be derived from alpha olefins, alkylene oxides, acetals, urethanes, etc.

The elastomeric block component of the copolymer may be isoprene or butadiene which may or may not be hydrogenated. This hydrogenation may be either partial or substantially complete. Selected conditions may be employed for example to hydrogenate the elastomeric block while not so modifying the vinyl arene polymer blocks. Other conditions may be chosen to hydrogenate substantially uniformly along the polymer chain, both the elastomeric and non-elastomeric blocks thereof being hydrogenated to practically the same extent, which may be either partial or substantially complete.

Typical of the rubbery block copolymers useful herein are the polystyrene-polybutadiene-polystyrene, polystyrene-polyisoprene-polystyrene and e.g., polystyrene-poly-(ethylenebutylene)-polystyrene and polystyrene-poly-(ethylenepropylene)-polystyrene. These copolymers may be prepared using methods taught, for example, in U.S. Pat. Nos. 3,239,478; 3,427,269; 3,700,633; 3,753,936; and 3,932,327. Alternatively, they may be obtained from Shell Chemical Co. under the trademarks Kraton 1101, 1102, 1107, 1650, 1652 and 1657; from Enichem under the Europrene Sol-T tradenames; and from Firestone under the tradename Stereon 840A.

Ethylene containing polymers are also commonly used for disposable applications and can be fragranced by the addition thereto of the fragrance compositions in accordance with the teachings of the invention. The adhesive of the invention may comprise at least one ethylene copolymer, and may comprise a blend of two or more polymers. The term ethylene copolymer, as used herein, refers to homopolymers, copolymers and terpolymers of ethylene. Examples of ethylene copolymers include copolymers with one or more polar monomers which can copolymerize with ethylene, such as vinyl acetate or other vinyl esters of monocarboxylic acids, or acrylic or methacrylic acid or their esters with methanol, ethanol or other alcohols. Included are ethylene vinyl acetate, ethylene methyl acrylate, ethylene n-butyl acrylate, ethylene acrylic acid, ethylene methacrylate and mixtures and blends thereof. Random and block copolymers, as well as blends thereof may be used in the practice of the invention.

Other adhesive compositions may be prepared according to the invention using, as a base polymer, amorphous polyolefins or blends thereof. Amorphous polyolefins are made by the stereospecific polymerization of polypropylene. Suitable commercial products include Eastman's P 1010. Copolymers of amorphous polypropylene and ethylene, amorphous polypropylene and butene and amorphous polypropylene and hexene are suitable as a base polymer, as are terpolymers of propylene, butene and ethylene. Commercial examples include Rextac 2315 (copolymer of amorphous polypropylene and ethylene) available from Rexene, Rextac 2730 (copolymer of amorphous polypropylene and butene) also available from Rexene and Vestoplast 750 and 708 (terpolymers of amorphous propylene, butene and ethylene) available from Huls.

Blends of any of the above base materials, such as blends of ethylene n-butyl acrylate and ethylene vinyl acetate and ethylene vinyl acetate and atactic polypropylene may also be used to prepare hot melt adhesive compositions. In all cases, the adhesives may be formulated with tackifying resins, plasticizers, waxes and/or other conventional additives in varying amounts as are known to those skilled in the art and as required for particular formulations, e.g., a pressure sensitive adhesive formulation.

Tackifying resins useful in the adhesive compositions of this invention include hydrocarbon resins, synthetic polyterpenes, rosin esters, natural terpenes, and the like. More particularly, and depending upon the particular base polymer, the useful tackifying resins may include any compatible resins or mixtures thereof such as natural and modified rosins including, for example, as gum rosin, wood rosin, tall oil rosin, distilled rosin, hydrogenated rosin, dimerized rosin, and polymerized rosin; glycerol and pentaerythritol esters of natural and modified rosins, including, for example as the glycerol ester of pale, wood rosin, the glycerol ester of hydrogenated rosin, the glycerol ester of polymerized rosin, the pentaerythritol ester of hydrogenated rosin, and the phenolic-modified pentaerythritol ester of rosin; copolymers and terpolymers of natured terpenes, including, for example, styrene/terpene and alpha methyl styrene/terpene; polyterpene resins having a softening point, as determined by ASTM method E28-58T, of from about 80° C. to 150° C.; phenolic modified terpene resins and hydrogenated derivatives thereof including, for example, the resin product resulting from the condensation, in an acidic medium, of a bicyclic terpene and a phenol; aliphatic petroleum hydrocarbon resins having a Ball and Ring softening point of from about 70° C. to 135° C.; aromatic petroleum hydrocarbon resins and the hydrogenated derivatives thereof; and alicyclic petroleum hydrocarbon resins and the hydrogenated derivatives thereof. Mixtures of two or more of the above described tackifying resins may be required for some formulations.

Various plasticizing or extending oils are also present in the composition in amounts of 5% to about 30%, preferably 5 to 25%, by weight in order to provide wetting action and/or viscosity control. Even higher levels may be used in cases where block copolymer containing hydrogenated mid-block are employed as the adhesive base polymer. The above broadly includes not only the usual plasticizing oils but also contemplates the use of olefin oligomers and low molecular weight polymers as well as vegetable and animal oil and their derivatives. The petroleum derived oils which may be employed are relatively high boiling materials containing only a minor proportion of aromatic hydrocarbons (preferably less than 30% and, more particularly, less than 15% by weight of the oil). Alternatively, the oil may be totally non-aromatic. The oligomers may be polypropylenes, polybutenes, hydrogenated polyisoprene, hydrogenated polybutadiene, or the like having average molecular weights between about 350 and about 10,000. Vegetable and animal oils include glyceryl esters of the usual fatty acids and polymerization products thereof.

Various petroleum derived waxes may also be used in amounts less than about 15% by weight of the composition in order to impart fluidity in the molten condition of the adhesive and flexibility to the set adhesive, and to serve as a wetting agent for bonding cellulosic fibers. The term “petroleum derived wax” includes both paraffin and microcrystalline waxes having melting points within the range of 130° F. to 225° F. as well as synthetic waxes such as low molecular weight polyethylene or Fisher-Tropsch waxes.

An antioxidant or stabilizer may also be included in the adhesive compositions described herein in amounts of up to about 3% by weight. Among the applicable antioxidants or stabilizers are high molecular weight hindered phenols and multifunctional phenols such as sulfur and phosphorous-containing phenols. Representative hindered phenols include: 1,3,5-trimethyl 2,4,6-tris (3,5-di-tert-butyl-4-hydroxy-benzyl)benzene; pentaerythritol tetrakis-3(3,5-di-tert-butyl-4-hydroxyphenyl)-propionate; n-octadecyl-3,5-di-tert-butyl-4-hydroxyphenol)-propionate; 4,4′-methylenebis (2,6-tert-butylphenol); 4,4′-thiobis (6-tert-butyl-o-cresol); 2,6-di-tertbutylphenol; 6-(4-hydroxyphenoxy)-2,4-bis(n-octyl-thio)-1,3,5-triazine; di-n-octadecyl 3,5-di-tert-butyl-4-hydroxy-benzyl-phosphonate; 2n-octylthio)-ethyl 3,5-di-tert-butyl-4hydroxy-benzoate and sorbitol hexa[3-(3,5-ditert-butyl-4-hydroxyphenyl)-propionate].

Other additives conventionally used in hot melt adhesives to satisfy different properties and meet specific application requirements also may be added to the adhesive composition of this invention. Such additives include, for example, fillers, pigments, flow modifiers, dyestuffs, which may be incorporated in minor or larger amounts into the adhesive formulation, depending on the purpose.

Hot melt adhesives may be prepared using techniques known in the art. Typically, the adhesive compositions are prepared by blending the components in the melt at a temperature of about 100 to 200° C. until a homogeneous blend is obtained, approximately two hours. Various methods of blending are known and any method that produces a homogeneous blend is satisfactory. The fragrance composition may be added, with stirring, any time during the preparation of the hot melt adhesive. The fragrance is added in an amount effective to be noticeable, and remain noticeable, to the user of the adhesive following cure and to the user of any end product manufactured using the scented adhesive of the invention. Typically, the fragrance will be present in an amount of about 0.001 to about 1.0 parts, preferably about 0.05 to about 0.15 parts, per 100 parts of the adhesive composition.

The resulting adhesives of the present invention are characterized by their ability to provide a fragrance and a durable bond to a nonwoven or tissue article and otherwise meet the unique requirements of the application, such flexibility, non-staining, and machinable viscosity.

The adhesive is applied to a substrate while in its molten state and cooled to harden the adhesive layer. The adhesive product can be applied to a substrate such as a nonwoven article by a variety of methods including coating or spraying in an amount sufficient to cause the article to adhere to another substrate such as tissue, nonwoven, or an unrelated material such as a low density polyolefin or other conventionally employed substrates.

### EXAMPLES

The following examples illustrate the production of suitable fragranced hot melt adhesives. In the examples, all parts are by weight and all temperatures in degree Celsius unless otherwise noted. Test procedures used herein are as follows:

Test Procedures

Viscosity

The viscosity as used herein is a Brookfield viscosity measured using a Brookfield viscometer model No. DV-II with spindle no. 27 at 20 rpm.

Color

Color was determined by visual inspection using a Gardner Color scale. (G1-G8)

Tensile Strength

Tensile Strength (Ultimate & Yield) was determined using dumbbell hot melt moulds and an Instron Unit.

Mettler Ring & Ball Softening Point

This test method describes the method used to determine the softening point of a thermoplastic composition in the range 50-150° C. using a Mettler Ring & Ball Assembly.

Odor

Odor was determined using a comparison test between a control sample with no fragrance and a fragranced sample. The odor is described by it intensity and smell.

Peel Strength

Peel strength was measured using T-peel bonds on an Instron unit at 12″/min. Bonds were made between polyethylene film and polypropylene nonwoven using a high speed laminator.

### Example 1

A hot melt adhesive composition comprising 22 parts adhesive polymer (Stereon 840A available from Firestone), 20 parts mineral oil (Witco), 58 parts of a tackifying resin (Escorez 5600 available from ExxonMobil), and 0.3 of an antioxidant (Ciba) was formulated using techniques known in the art. An exemplary procedure involves placing approximately half of the total tackifying resin in a jacketed mixing kettle, which is equipped with rotors, and raising the temperature to a range from about 100° C. to 200° C. The precise temperature utilized depends on the softening point of the particular tackifying resin. When the resin has melted, stirring is initiated and the block polymer and stabilizer are added together with any optional additives whose presence may be desired. Mixing and heating are continued until a smooth homogeneous mass is obtained.

### Example 2

The rubber based hot melt adhesive was prepared in accordance with Example land 0.1% by weight of Spicy Oriental Amber Powder (Quest Fragrance Q-26257) was added thereto with stirring.

Viscosity, color, tensile strength (ultimate and yield) and Ring & Ball tests were carried out on the composition of Examples 1 and 2 immediately after completion of stirring. Thereafter, the adhesives were placed in an oven at 170° C. for approximately 72 hours and tested for the presence of odor. The samples were then removed from the oven and allowed to cool to ambient conditions. The samples were covered overnight and then evaluated for odor type and intensity. A description of the intensity and smell of each sample was recorded. Results are shown in Table 1.

TABLE 1

| | Example 1 | Example 2 |
|---|---|---|
| VISCOSITY @ 325 F. | 2890 cps | 2920 cps |
| COLOR | G1 | G1 |
| TENSILE STRENGTH | | |
| Yield (psi) | 5.9 | 5.1 |
| Ulitmate (psi) | 104 | 110 |
| RING & BALL | 75.7 | 75.0 |
| AGED ODOR | Moderate | Moderate baby |
| | Resin/Oil | powder fragrance |
| T-Peel max (g/linear inch) | | |
| Initial | 190 | 150 |
| 1 week at 40° C. | 120 | 133 |

The results indicate that after exposure to elevated temperatures for extended periods of time, the presence of the fragrance is still noticeable.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A hot melt adhesive comprising a non-encapsulated scented material.

2. The adhesive of claim 1 wherein the scented material has a flashpoint of greater than 100° F.

3. The adhesive of claim 2 wherein the scented material has a flashpoint of greater than 200° F.

4. The adhesive of claim 3 wherein the scented material is Spicy Oriental Amber Powder (Quest Fragrance Q-26257).

5. The adhesive of claim 1 which is a pressure sensitive adhesive.

6. The adhesive of claim 1 which is a low application temperature hot melt adhesive.

7. A disposable absorbent article comprising a liquid-permeable topsheet, a liquid-impermeable backsheet, a fluid-absorbent core material positioned between the topsheet and the backsheet and a hot melt adhesive containing a nonencapsulated scented material.

8. The article of claim 7 wherein the scented material has a closed cup flashpoint of greater than 100° F.

9. The article of claim 8 wherein the scented material has a closed cup flashpoint of greater than 200° F.

10. The article of claim 9 wherein the scented material is Spicy Oriental Amber Powder (Quest Fragrance Q-26257).

11. The article of claim 7 wherein the fluid absorbent core material comprises a nonwoven fabric.

12. The article of claim 7 wherein the backsheet is attached to the topsheet.

13. The article of claim 7 which is a diaper, a feminine hygiene product or adult incontinent product.

14. The article of claim 7 wherein the hot melt adhesive is a pressure sensitive hot melt adhesive.

15. The article of claim 7 wherein the hot melt adhesive is a low application temperature hot melt adhesive.

16. A process for bonding a tissue or a nonwoven substrate to similar or dissimilar substrates in the construction of a disposable absorbent product comprising applying to at least one substrate a molten hot melt adhesive composition and bonding said substrates together, wherein said hot melt adhesive comprises a non-encapsulated scented material.

17. The process of claim 16 wherein the scented material has a closed cup flashpoint of greater than 100° F.

18. The process of claim 17 wherein the scented material has a closed cup flashpoint of greater than 200° F.

19. The process of claim 18 wherein the scented material is Spicy Oriental Amber Powder (Quest Fragrance Q-26257).

20. The process of claim 16 wherein the hot melt adhesive is a low application temperature hot melt adhesive.
